(19) United States
(12) Patent Application Publication
(10) Pub. No.: US 2021/0322602 A1
(43) Pub. Date: Oct. 21, 2021

(54) ENCLOSURE FOR ERADICATING BACTERIA AND VIRUSES

(71) Applicant: Ray SC LLC, West Palm Beach, FL (US)

(72) Inventor: Salvatore Furia, Rydal, PA (US)

(21) Appl. No.: 17/236,459

(22) Filed: Apr. 21, 2021

Related U.S. Application Data
(60) Provisional application No. 63/013,157, filed on Apr. 21, 2020.

(51) Int. Cl. A61L 2/10; A61L 2/26

(52) U.S. Cl. CPC A61L 2/10; A61L 2/26; A61L 2202/14; A61L 2202/20; A61L 2202/121; A61L 2202/122

(57) ABSTRACT

An enclosure for eradicating bacteria and viruses. The enclosure includes a bottom, sides, and a top, creating an interior space to store objects. Each of the bottom, the sides, and the top include a UV light array that emits UV-C light to eradicate bacteria and viruses. Since the enclosure is configured to allow objects to be placed directly on the floor of the of the interior space, the bottom UV light array may be set back from the floor of the interior space to allow the UV-C light to disperse and cover the entire bottom surface of the objects. Specifically, a skeletal structure of the bottom UV light array may support a protective film above the array of UV light sources to create a negative space between the protective film and the array of UV light sources. The UV light sources emit UV-C light across a wide angle θ (e.g., 120 degrees or more). The wide angle θ of the UV-C light and the negative space allow the UV-C light from the bottom array to overlap and emit through the entire surface of the protective film.

### CROSS REFERENCE TO RELATED APPLICATIONS

This application claims priority to U.S. Prov. Pat. Appl. No. 63/013,157, filed Apr. 21, 2020, which is hereby incorporated by reference.

### BACKGROUND

Electronic devices, such as smartphones and tablets, are known carriers of microbes and viruses. Various studies have even found E. coli, streptococcus, and Methicillin-resistant Staphylococcus aureus (MRSA) living on mobile phones and tablets. Other objects, such as keys, may also carry bacteria and viruses.

Ultraviolet C (UV-C) light is highly effective at killing bacteria and viruses by destroying the molecular bonds that hold their DNA together. Broad-spectrum germicidal UV-C light, which has wavelengths between 224 and 285 nanometers (nm), is particularly effective. Accordingly, conventional UV-C light is routinely used to decontaminate surgical equipment.

Several prior art enclosures purport to use UV light to eradicate bacteria and virus from the surfaces of electronic devices. However, those prior art enclosures have technical drawbacks that prevent the enclosure from eradicating bacteria and viruses from all exterior surfaces of the electronic devices, require the electronic devices to remain within the enclosure for longer than is preferable, damage the sensitive touchscreens of electronic devices, and may even expose users to the UV light, which is a human health hazard that can cause skin cancer and cataracts.

Accordingly, there is an improved enclosure uses ultraviolet UV-C light to eradicate bacteria and viruses living on electronic devices and other objects.

### SUMMARY

In order to overcome those and other drawbacks of the prior art, an enclosure for eradicating bacteria and viruses is provided. The enclosure includes a bottom, sides, and a top, creating an interior space to store objects. Each of the bottom, the sides, and the top include a UV light array that emits UV-C light and eradicate bacteria and viruses.

Since the enclosure is configured to allow objects to be placed directly on the floor of the of the interior space, the bottom UV light array may be set back from the floor of the interior space to allow the UV-C light to disperse and cover the entire bottom surface of the objects. Specifically, a skeletal structure of the bottom UV light array may support a protective film above the array of UV light sources to create a negative space between the protective film and the array of UV light sources. The UV light sources emit UV-C light across a wide angle θ (e.g., 120 degrees or more). The wide angle θ of the UV-C light and the negative space allow the UV-C light from the bottom array to overlap and emit through the entire surface of the protective film.

### BRIEF DESCRIPTION OF THE DRAWINGS

The accompanying drawings are incorporated in and constitute a part of this specification. Features in the accompanying drawings are illustrated for clarity and are not necessary drawn to scale. It is to be understood that the drawings illustrate only some examples of the disclosure and other examples or combinations of various examples that are not specifically illustrated in the figures may still fall within the scope of this disclosure. Examples will now be described with additional detail through the use of the drawings.

FIG. 1 is an exterior view of an enclosure for eradicating bacteria and viruses using ultraviolet C (UV-C) light according to an exemplary embodiment.

FIG. 2 is an interior view of the enclosure according to an exemplary embodiment.

FIG. 3 is another interior view of the interior space of the enclosure according to exemplary embodiment.

FIG. 4 is a side view of the enclosure according to exemplary embodiment.

FIGS. 5A and 5B are views of a side or top UV light array according to an exemplary embodiment.

FIG. 6 is a side view of a bottom UV light array according to an exemplary embodiment.

FIG. 7 is a block diagram of the enclosure according to an exemplary embodiment.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

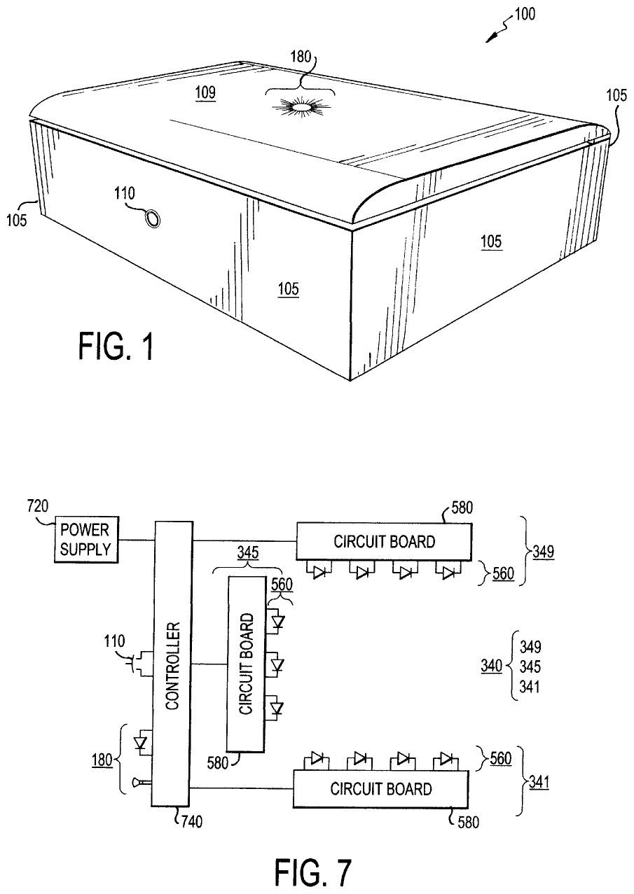

FIG. 1 is an exterior view of an enclosure 100 for eradicating bacteria and viruses using ultraviolet C (UV-C) light according to an exemplary embodiment.

As shown in FIG. 1, the enclosure includes a bottom 101, sides 105, a top 109, an on/off button 110, and an output device 180. As shown in the enclosed figures, the enclosure 100 may be a rectangular box with four sides 105 (and a bottom 101 and a top 109). However, the enclosure 100 may be any shape (e.g., cylindrical).

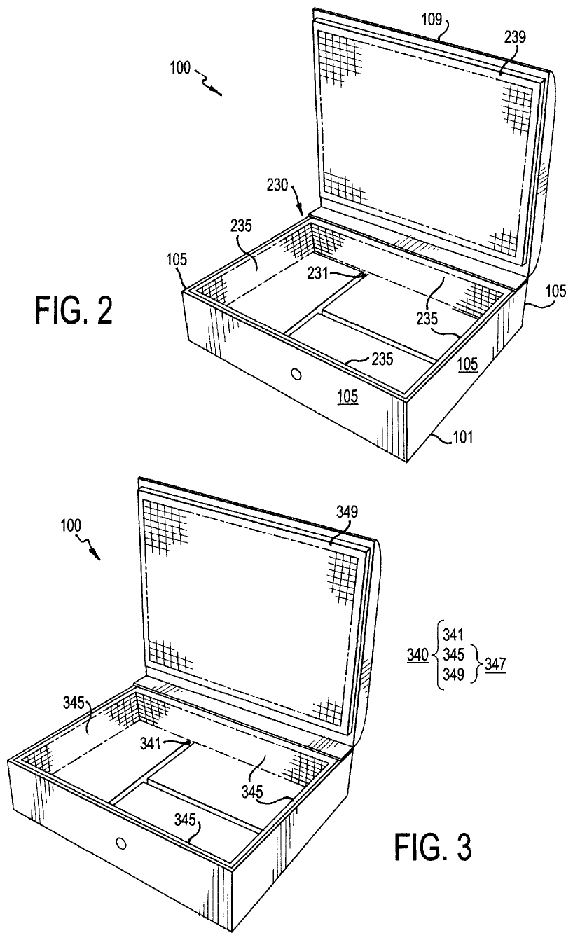

FIG. 2 is an interior view of the enclosure 100 according to an exemplary embodiment.

As shown in FIG. 2, the bottom 101 of the enclosure 100 forms a floor 231, the sides 105 of the enclosure 100 form interior walls 235, and the top 109 of the enclosure 100 forms a ceiling 239. The floor 231, the walls 235, and the ceiling 239 form an interior space 230 of the enclosure 100 bounded by the floor 231, the walls 235, and the ceiling 239.

FIG. 3 is another interior view of the interior space 230 of the enclosure 100 according to exemplary embodiment.

As shown in FIG. 3, each of the interior walls of the interior space 230 includes a UV light array 340. As a result, all of the exterior surfaces of an object 201, such as electronic device, within the interior space 230 of the enclosure 100 are exposed to UV-C light. For example, the floor 231 of the interior space 230 includes a bottom UV light array 341 that emits UV-C light upward in a direction substantially orthogonal to the floor 231 of the interior space 230. Each side wall 135 of the interior space 230 may include a side UV light array 345 that emits UV-C light in a direction substantially orthogonal to the light emitted by the bottom UV light array 231. And the ceiling 239 of the interior space 230 may include a top UV light array 349 that emits UV-C light in a direction substantially orthogonal to the light emitted by each of the side UV light arrays 345 and substantially opposite the light emitted by the bottom UV light array 341.

One of the sides 105 or top 109 of the enclosure 100 opens to provide access to the interior space 230 and closes to prevent UV-C light emitted within the interior space 230 from exiting the interior space 230. As shown in FIGS. 2 and 3, the top 109 of the enclosure 100 may be a lid that allows one or more objects 201 to be placed into the interior space 230 of the enclosure 100 from above. However, in other embodiments, a side 105 of the enclosure 100 may be a door that opens to allow one or more objects 201 to be placed into the interior space 230 of the enclosure 100 from the side 105. In either of those embodiments, the lid or door includes a side UV light array 345 or a top UV light array 349 so that all of the exterior surfaces of an object 201 within the interior space 230 of the enclosure 100 are exposed to UV-C light.

In most embodiments, the side UV light arrays 345 and the top UV light array 349 on the walls 235 and the ceiling 239 of the interior space 340 are largely similar. Accordingly, to differentiate the side UV light arrays 345 and the top UV light array 349 from the bottom UV light array 341, the side UV light arrays 345 and the top UV light array 349 are referred to herein as additional UV light arrays 347.

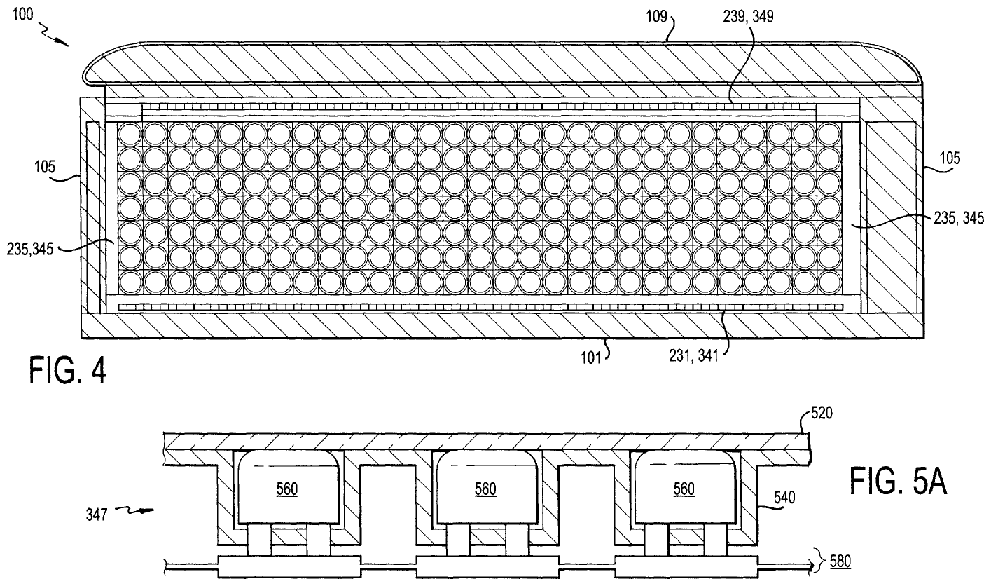

FIG. 4 is a side view of the enclosure 100 according to exemplary embodiment.

As shown in FIG. 4, the bottom 101 of the enclosure 100 creates a floor 231 of the interior space 230. Meanwhile, each side 105 of the enclosure 100 creates an interior wall 235 and the top 109 creates a ceiling 239. Each of the floor 231, the walls 235, and the ceiling 239 includes a UV light array 340. The floor 231 includes a bottom UV light array 341; each wall 235 includes a side UV light array 345; and the ceiling 239 includes a top UV light array 349.

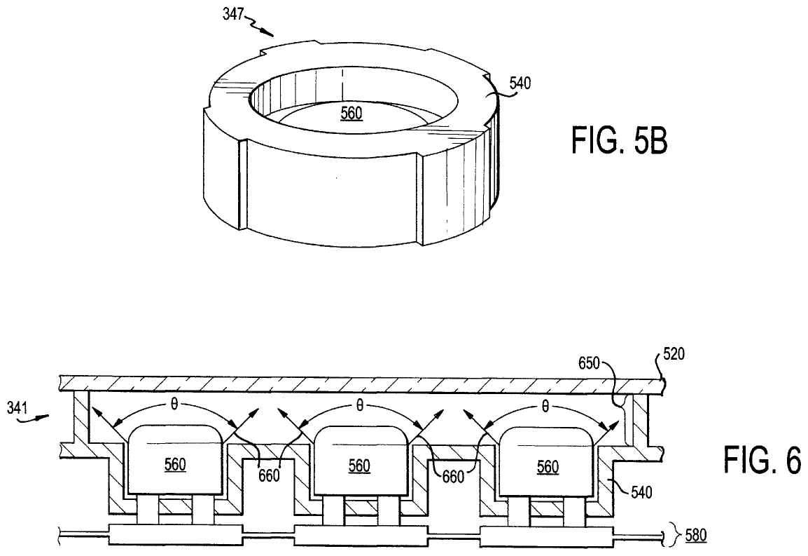

FIGS. 5A and 5B are views of the one of the additional UV light arrays 347 (i.e., one of the side UV light arrays 345 or the top UV light array 349) according to an exemplary embodiment.

As shown in FIG. 5A, each additional UV light array 347 may include a protective film or silicone mesh 520, a skeletal structure 540, an array of UV light sources 560, and a circuit board 580. While only one UV light source 560 is shown for simplicity, each additional UV light array 347 may include any number of UV light sources 560. The UV light sources 560 may be arranged in rows and columns or in any tessellation (e.g., a honeycomb pattern, etc.).

The UV light sources 560 may be light emitting diodes (LEDs). Alternatively, the UV light sources 560 may include one or more UV tube lamps and computer-controlled windows (similar to a liquid crystal display). The UV light sources 560 may emit light having a wavelength between 200 and 300 nanometers (nm). Preferably, the UV lights emit light having a wavelength between 224 and 285 nm. The UV light sources 560 may emit light across an angle of 120 degrees or more. The flux power of the UV light sources 560 may be at least 10 milliwatts (mW). Preferably, the flux power of the UV light sources 560 are at least 30 mW (e.g., 30-40 mW). Higher power UV light sources 560 reduce the amount of time that the objects 201 need to be placed in the enclosure. However, driving the UV light sources 560 with higher power reduces the life span of those UV light sources 560. Accordingly, each UV light array 340 (or individual UV light source 560) may be replaceable.

As shown in FIGS. 5A and 5B, the skeletal structure 540 surrounds each of the UV light sources 560 and provides a structure that protects the UV light sources 560.

The protective film or silicone mesh 520, which is attached to skeletal structure 540, is situated between the UV light sources 560 and the interior space 260 of the enclosure 100. The protective film or silicone mesh 520 is transparent to the UV-C light.

The circuit board 580 is coupled to each of the UV light sources 560 to control each of the UV light sources 560 as described below.

FIG. 6 is a side view of the bottom UV light array 341 according to an exemplary embodiment. Similar to the additional UV light array 347, the bottom UV light array 341 may include a protective film or silicon mesh 520, a skeletal structure 540, an array of UV light sources 560, and a circuit board 580. Again, while only three UV light sources 560 are shown for simplicity, the bottom UV light array 341 any number of UV light source 560. The UV light sources 560 arranged in any pattern.

Because the enclosure 100 is configured to allow objects 201 to be placed directly on the floor 231 of the of the interior space 230 as shown in FIG. 2, the bottom UV light array 341 may be set back from the floor 231 of the interior space 230 to allow the UV-C light to disperse and cover the entire bottom surface of the objects 201. Therefore, unlike the additional UV light arrays 347, the skeletal structure 540 of the bottom UV light array 341 supports the protective film 520 above the array of UV light sources 560 to create a negative space 650 between the protective film 520 and the array of UV light sources 560. The UV light sources 560 emit UV-C light 660 across a wide angle θ (e.g., 120 degrees or more). As shown in FIG. 6, the wide angle θ of the UV-C light 660 and the negative space 650 allow the UV-C light 660 from the UV light sources 560 of the bottom array 341 to overlap and emit through the entire surface of the protective film 520.

Referring back to FIG. 1, the enclosure 100 may include an on/off button 110 and an output device 180.

The on/off button 110 may be any mechanism selectable by a user to initiate or abort the process of eradicating bacteria and viruses within the interior space 230 of the enclosure 100 using UV-C light.

The output device 180 may be any device outputs an indication that the predetermined time period is ongoing and/or that the predetermined time period is complete. The output device 180 may output a visual indication, an audible indication, a tactile indication, etc. As shown in the drawings, the output device may be LED lights that output a visual indication of the UV emission process and/or an indication that the predetermined time period is complete. Additionally or alternatively, the output device may include a speaker that outputs an audible indication of the UV emission process (e.g., a song that plays until the process is complete) and/or an audible indication that the predetermined time period is complete (i.e., a sound indicating that the process is complete).

FIG. 7 is a block diagram of the enclosure 100 according to an exemplary embodiment. In the example shown in FIG. 7, the enclosure 100 includes a top UV light array 349, one side UV light array 345, and a bottom UV light array 345. However, as described above, the enclosure 100 may include any number of side UV light arrays 345.

As shown in FIG. 7, each of the UV light arrays 340 is coupled to a controller 740, which is coupled to a power supply 720. The power supply 720 may include a battery and/or a wired connection to a power source. The battery may be rechargeable. If the power supply 720 includes a battery, the power supply may include an output device that outputs an indication (e.g., a visual indication) of the battery charge. The controller 740 may be any processing unit that controls the UV light arrays 340 to perform the functions described herein. The controller 740 may be an integrated circuit, a state machine, etc.

In the example shown in FIG. 7, each UV light array 340 includes a circuit board 580 coupled to each UV light source 560. However, as one of ordinary skill in the art will recognize, the UV light sources 560 of multiple UV light arrays 340 may be coupled to the same circuit board 580.

In some embodiments, the enclosure 100 is configured to output UV light for a predetermined time period that has been determined to be sufficient to eradicate a sufficient amount (e.g., 99 percent or 99.9 percent) of the bacteria and viruses on the exterior surfaces of the objects 201. Accordingly, the controller 740 may supply power to the UV light arrays 340 for the predetermined time period. By using UV lights 560 with sufficient flux power and emitting UV-C light 660 from all of the walls of the interior space 230, the enclosure 100 may require a predetermined time period as short as 10-15 seconds.

Some electronic devices have touchscreen displays that are sensitive to prolonged exposure to UV-C light. Accordingly, users may be instructed to place touchscreen devices into the enclosure 100 facing a certain direction (for example, with the touchscreen display facing down) and the controller 740 may cause one of the UV light arrays 340 (in that example, the bottom UV light array 341) to emit UV-C light for a shorter predetermined time period (e.g., 10 seconds) and cause the other UV light arrays 340 to emit UV-C light for a longer predetermined time period (e.g., 10 seconds).

Because exposure to UV-C light may be a health hazard, the lid or door of the enclosure 100 may be locked electronically (e.g., by the controller 740) when the UV emission process starts and may be unlocked (e.g., by the controller 740) only after the predetermined time period. Alternatively, the lid or door may include a sensor that detects when the lid or door is open and the controller 740 may turn off the UV light arrays 340 in response to a determination that the lid or door is open.

In some embodiments, the enclosure 100 may be configured to output information to an external device. For example, the enclosure 100 may communicate with a local area network (via a wired or wireless connection) and output information to a user device (e.g., a smartphone, a tablet, a personal computer, etc.). In another example, the enclosure 100 may output information to a server (e.g., via the local area network and the internet), which may provide the information to user devices (e.g., via a smartphone application, a website, etc.) The enclosure 100 may output information indicative of the battery charge of the power supply 720, a notification that the battery of the power supply 720 needs to be recharged, information indicative of the lifespan of the UV lights 560, a notification that one or more of the UV lights 560 are approaching the end of their estimated life span, etc.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. An enclosure for eradicating bacteria and viruses using ultraviolet C (UV-C) light, the enclosure comprising:
a bottom that creates a floor of an interior space, the floor of the interior space comprising:
a bottom array of UV lights that emit UV light;
a protective film configured to support objects above the bottom array of UV lights, the protective film being transparent to the UV light;
a skeletal structure that supports the protective film above the bottom array of UV lights to create a negative space between the protective film and the bottom array of UV lights, the negative space having a height such that UV light from a plurality of UV lights overlaps and emits through the surface of the protective film;
side walls that encircle an interior space, each of the side walls including an additional array of UV lights that emit UV light; and
a top that covers the interior space, the including an additional array of UV lights that emit UV light,
wherein the top or one of the side walls comprises a lid or door that opens to provide access to the interior space and closes to prevent UV light emitted within the interior space from exiting the interior space.

2. The enclosure of claim 1, wherein the UV lights are light emitting diodes (LEDs).

3. The enclosure of claim 2, wherein the UV lights emit light having a wavelength between 260 and 280 nanometers.

4. The enclosure of claim 2, wherein the UV lights emit light having a wavelength between 260 and 265 nanometers.

5. The enclosure of claim 2, wherein the light emitting diodes have a flux power of at least 10 milliwatts (mW).

6. The enclosure of claim 2, wherein the light emitting diodes have a flux power between 30 and 40 mW.

7. The enclosure of claim 1, wherein the UV lights of the bottom array each emit UV light at an angle of at least 120 degrees.

8. The enclosure of claim 1, wherein the bottom array of UV lights emits UV light for a first predetermined time period and each additional array of UV lights emit UV light for a second predetermined time period that is longer than the first predetermined time period.

9. The enclosure of claim 1, wherein the lid or door is configured to remain locked while the bottom array of UV lights or the additional arrays of UV lights emit UV light.

10. The enclosure of claim 1, wherein:
the lid or door includes a sensor; and
the bottom UV light array and the additional UV light arrays are configured to turn off in response to a determination that the lid or door is open.

11. A method of eradicating bacteria and viruses using ultraviolet C (UV-C) light, the method comprising:
providing an enclosure comprising:
a bottom that creates a floor of an interior space, the floor of the interior space comprising:
a bottom array of UV lights that emit UV light;
a protective film configured to support objects above the bottom array of UV lights, the protective film being transparent to the UV light; and
a skeletal structure that supports the protective film above the bottom array of UV lights to create a negative space between the protective film and the bottom array of UV lights, the negative space having a height such that UV light from a plurality of UV lights overlaps and emits through the surface of the protective film;
side walls that encircle an interior space, each of the side walls including an additional array of UV lights that emit UV light; and
a top that covers the interior space, the including an additional array of UV lights that emit UV light;
eradicating bacteria and viruses by emitting UV light from the bottom array of UV lights and each addition array of UV lights.

12. The method of claim 11, wherein the UV lights are light emitting diodes (LEDs).

13. The method of claim 12, wherein the UV lights emit light having a wavelength between 200 and 300 nanometers.

14. The method of claim 12, wherein the UV lights emit light having a wavelength between 224 and 285 nanometers.

15. The method of claim 12, wherein the light emitting diodes have a flux power of at least 10 milliwatts (mW).

16. The method of claim 2, wherein the light emitting diodes have a flux power between 30 and 40 mW.

17. The method of claim 11, wherein the UV lights of the bottom array each emit UV light at an angle of at least 120 degrees.

18. The method of claim 11, wherein the bottom array of UV lights emits UV light for a first predetermined time period and each additional array of UV lights emit UV light for a second predetermined time period that is longer than the first predetermined time period.

19. The method of claim 11, wherein the lid or door is configured to remain locked while the bottom array of UV lights or the additional array of UV lights emit UV light.

20. The method of claim 11, wherein the lid or door includes a sensor and the method further comprises:
turning off the bottom array of UV lights and the additional arrays of UV lights in response to a determination that the lid or door is open.
